(19) United States
(12) Patent Application Publication
(10) Pub. No.: US 2007/0106174 A1
(43) Pub. Date: May 10, 2007

(54) Intrauterine anesthetic applicator and cell collection device and method of use

(75) Inventors: Gerald J. Sanders, Sonoma, CA (US); Gerald Feuer, Atlanta, GA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSPEC LLC, San Francisco, CA (US)

(21) Appl. No.: 11/245,498

(22) Filed: Oct. 7, 2005

(51) Int. Cl. A61B 10/00; A61M 1/00; A61M 5/178

(52) U.S. Cl. 600/563; 600/562; 604/27; 604/35; 604/38

(57) ABSTRACT

Apparatus and methods are provided for applying anesthetic or other medication to a desired location within a patient's uterus and for collecting cells, wherein an inner tube is configured to reciprocate through the lumen of an outer tube. The outer tube includes a foam applicator that facilitates delivery of anesthetic to the uterus prior to the cell collection procedure.

### FIELD OF THE INVENTION

The present invention relates to apparatus and methods for anesthetizing the uterus and collecting intrauterine cells for diagnostic purposes. More particularly, the present invention relates to the application of anesthetic and removal of a uterine tissue sample while reducing a patient's discomfort.

### BACKGROUND OF THE INVENTION

Various gynecological procedures require an analysis of intrauterine tissue. Previously-known devices typically have not been concerned with patient comfort as a design priority and may cause patient pain or discomfort during use. In addition, previously-known cell collection systems have required multiple devices to be inserted serially into the uterus to obtain a sample.

Intrauterine tissue collection apparatus and methods are known in which anesthetic is applied to the external cervical os using an elongated tube having a hard plastic acorn disposed at the distal end. The tube is inserted such that the acorn contacts the cervical os, and the anesthetic is then released. The acorn generally keeps the anesthetic localized at the application site, but some leakage may occur after the acorn is withdrawn. This leakage reduces the available anesthetic at the application site and thus the duration and intensity of the pain relief.

In addition, some patients are reluctant to undergo gynecological procedures due to the discomfort involved. For example, in U.S. Pat. No. 6,423,038 to Vancaillie, an intrauterine anesthesia device is described which is used to administer anesthetic and is then withdrawn to allow access for the next instrument used in the intra-uterine manipulation. To reduce patient discomfort, it would be desirable to provide apparatus device and methods that reduce the number of instruments that must be separately inserted into the patient.

Another source of discomfort associated with previously-known devices may arise due to the size of the device that must be inserted into the patient's vagina. For example, U.S. Pat. No. 5,231,992 to Leon describes a cervical cell collection device having a diameter of 40 to 52 mm. That device may by inserted and remain in the patient's cavity for 24 hours. To reduce a patient's discomfort, it would be desirable to provide a cell collection device with a reduced size compared to such previously known devices.

A further disadvantage of previously known apparatus is the amount of tissue removed during the cell collection process. U.S. Pat. No. 5,713,369 to Tao et al. describes a brush having a multiplicity of bristles designed to comprehensively contact the undilated uterine canal. It would therefore be desirable to provide apparatus and methods that reduce the degree of patient discomfort when obtaining a medically useful sample size.

### SUMMARY OF THE INVENTION

In light of the foregoing, it is an object of the present invention to provide apparatus and methods for use in applying anesthetic and collecting intrauterine cell samples that facilitates anesthetic delivery and reduces leakage during the cell collection process.

It is also an object of this invention to provide apparatus and methods for applying an anesthetic and collecting uterine cell samples that reduces the number of instruments that must be serially inserted and removed from the patient's cavity.

It is a further object of the present invention to provide apparatus and methods for applying anesthetic and collecting uterine cell samples that employs a compact device profile to reduce patient discomfort.

Another object of the present invention is to provide apparatus and methods for applying anesthetic and collecting uterine cell samples that uses suction to collect cells from a focal sample area.

These and other objects of the present invention are accomplished by providing apparatus comprising an elongated outer tube having an applicator mounted on the distal end for delivering anesthetic, and a cell collection tube configured to be advanced through the elongated outer tube. The apparatus of the present invention has a compact insertion profile that minimizes discomfort, and facilitates the routine use of anesthetic in connection with intrauterine sample collection. In addition, the device is configured to enable cell collection without serially insertion of multiple devices.

In the first preferred embodiment, the elongated outer tube comprises a polymer or other non-metallic material having a length of approximately 20 cm to 30 cm long and a diameter of 3 mm to 5 mm. The outer tube includes a lumen that is open at the distal end and fitted with a standard Luer-Lok adapter at the proximal end to permit coupling to a conventional syringe. An applicator, preferably conical and comprising a closed-cell foam rubber, is disposed on the distal end of the elongated outer tube.

The apparatus further comprises an inner tube, preferably is at least 5 cm to 10 cm longer than the outer tube, configured to freely reciprocate through the lumen of the outer tube. The inner tube has proximal and distal ends and a lumen extending therebetween. The lumen is closed at the distal end but includes an aperture disposed in a lateral wall surface that communicates with the collection lumen.

A plunger is disposed in sliding relation within the inner tube. The plunger includes a distal end having the size and shape approximating the interior dimensions of the inner tube, so that proximal displacement of the plunger causes intrauterine fluid and cells to be drawn through the aperture and into the collection lumen.

In an alternative embodiment, the plunger is omitted and the inner tube instead has a Luer-Lok fitting at its proximal end. This fitting permits a conventional syringe to be attached to the proximal end of the inner tube so that the collection lumen may be used to dispense anesthetic into the uterus and subsequently permit uterine fluids and cells to be aspirated through the collection lumen.

Methods of using the apparatus of the present invention to anesthetize a patient's uterus and to collect cell samples also are provided.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a side view depicting an exemplary embodiment of the apparatus of the present invention;

FIG. 2 is a side view of the individual components of the apparatus of FIG. 1;

FIG. 3 is a schematic view depicting a method of using the apparatus of FIGS. 1 and 2;

FIG. 4 is a side view depicting an alternative embodiment of the apparatus of the present invention; and

FIG. 5 is a side view depicting the individual components of the apparatus of FIG. 4.

### DETAILED DESCRIPTION OF THE INVENTION

The present invention is directed to apparatus and methods for applying anesthetic and collecting intrauterine tissue samples, while reducing patient discomfort. Previously-known intrauterine cell collection apparatus have focused primarily on efficient mechanisms for cell collection, with little regard to patient comfort. Consequently, most patients regard such devices as unpleasant, invasive and occasionally painful. The present invention is designed to address these issues.

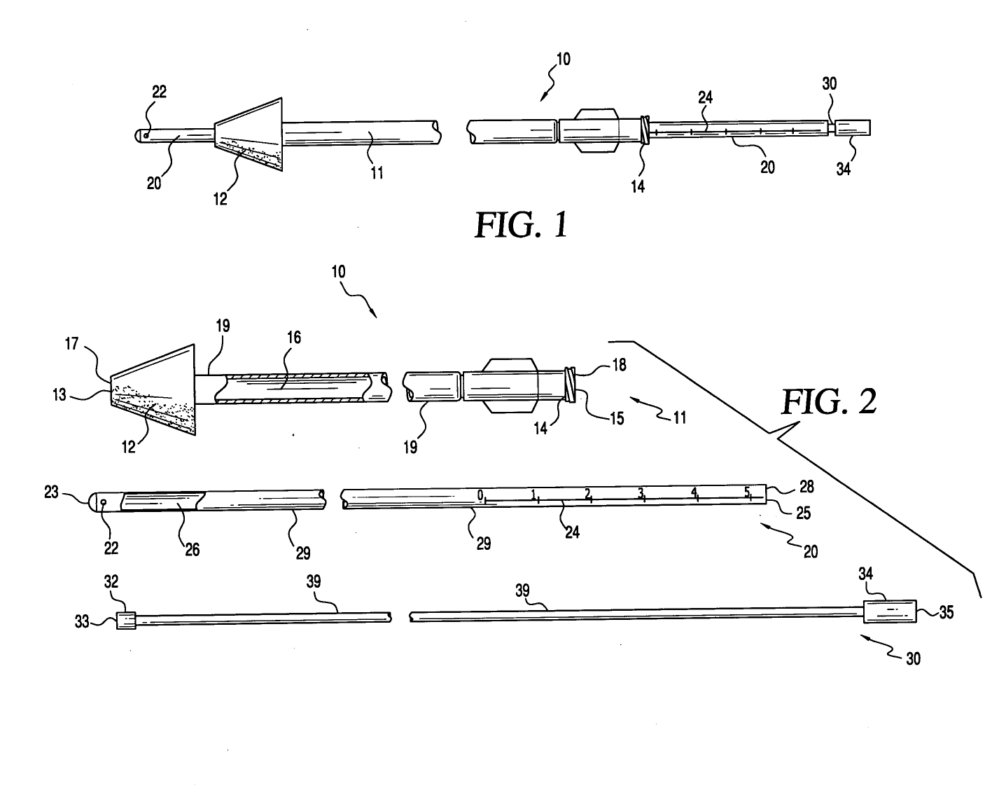

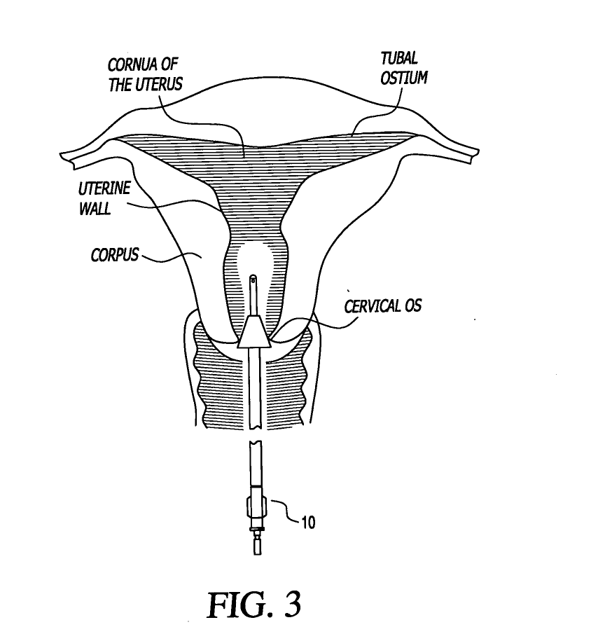

Referring to FIGS. 1 and 2, an exemplary embodiment of the apparatus of the present invention is described. Device 10 is configured to conduct all steps necessary for intrauterine tissue collection while substantially reducing patient discomfort, compared to previously-known methods and apparatus. In particular, device 10 permits both application of anesthetic and cell collection with only a single insertion and removal of the medical device. An illustrative embodiment of the device of the present invention is shown in an assembled state in FIG. 1; the individual components are depicted in FIG. 2.

Device 10 comprises three members: elongated outer tube 11, inner tube 20, and plunger 30. Elongated outer tube 11 has anesthetic applicator 12 disposed at distal end 13 and Luer-lok fitting 14 disposed on proximal end 15. Lumen 16 that extends from distal end 13 to proximal end 15 to provide communication between opening 18 of Luer-lok fitting 14 at the proximal end and opening 17 at the distal end.

Applicator 12 preferably comprises a conical resilient closed-cell foam material capable of conforming to the cervical os. Applicator 12 of device 10 differs from the hard plastic “acorns” used in previous-known devices in that foam cell is designed to conform to the anatomy of the cervical os, rather than deforming the anatomy to conform to the shape of the instrument. Applicator 12 is designed to remain in place against the cervical os during the duration of the sample collection procedure, thereby helping to deliver and retain anesthetic at that location.

Applicator 12 may be affixed to body 19 of outer tube 11 using any of a variety of methods, such as with biocompatible adhesive or interference fit. Applicator 12 preferably has a base diameter of approximately 15 mm and a height of approximately 15 mm. Other embodiments may vary in dimension to accommodate differences between patients.

Outer tube 11 preferably is essentially cylindrical and constructed of semi-rigid clear or opaque polymer, such as silicon, polyethylene, or polycarbonate, which allows visualization of the contents of lumen 16. Although the device will function sufficiently well if constructed of metal alloy or glass, the use of polymers is preferred to avoid patient discomfort associated with the “cold” feeling of metallic instruments. In a preferred embodiment, outer tube 11 has a length of about 20 cm to 30 cm and a diameter of about 3 mm to 5 mm.

Referring also to FIG. 2, inner tube 20 is described. Inner tube 20 preferably is cylindrical and has a length that is at least 5 cm to 10 cm longer than outer tube 11, and a diameter sufficient to permit the inner tube to be reciprocated inside lumen 16 of outer tube 11. Inner tube 20 includes lumen 26 that extends between opening 28 at proximal end 25 and tissue collection aperture 22 disposed near distal end 23. Although depicted in FIG. 2 as circular, aperture 22 may have other geometries or other features, such as flanges or angled edges, to facilitate removal of a tissue sample.

While distal end 23 is depicted as closed in FIGS. 1 and 2, in alternative embodiments distal end 23 may be removed by the clinician, thereby selectively allowing communication with lumen 26. Similarly, aperture 22 may be disposed at distal end 23, for example, where inner tube 20 further included a pull wire or other means to articulate the distal region of inner tube 20.

In a preferred embodiment, inner tube 20 has markings 24 on body 29 near proximal end 25 that enable the clinician to determine the circumferential position of aperture 22 relative to proximal end 25. Markings 24 also may be provided to indicate depth of overall insertion or insertion relative to outer tube 11.

Still referring to FIGS. 1 and 2, plunger 30 is configured to reciprocate within lumen 26 of inner tube 20. Plunger 30 comprises shaft 39 connecting end portion 32 at distal end 33 with handle 34 at proximal end 35. End portion 32 preferably is configured so that its exterior surface is similar in shape to the interior surface of lumen 26 of inner tube 20. Because end portion 32 of plunger 30 has a geometry that approximates lumen 16 of inner tube 11, proximal and distal displacement of end portion 32 within inner tube 11 creates negative and positive pressure differences in the volume of lumen 16 between end portion 32 and distal end 23 of inner tube 20. These pressure differences may be equalized with passage of air, tissue, or other matter through aperture 22.

Plunger 30 preferably comprises a single piece of molded polymer, although other embodiments may have multiple pieces formed of different material, such as a two-piece design with a rubber end piece 32 and a rigid wooden shaft 39, for example. Plunger 30 preferably extends beyond the proximal end of inner tube 20 sufficiently far to allow a clinician to comfortably use handle 34 to manipulate plunger 30. For example, plunger 30 may extend for a length of 2 cm to 3 cm beyond the proximal end of inner tube 11, although a shorter or longer length for plunger 30 may be desired in particular case.

Proximal end 35 of plunger 30 includes handle 34. Handle 34 may be simple in design, such as a continuation of shaft 39, or more complex, such as providing an increased diameter to facilitate grip, as depicted in FIGS. 1 and 2. Preferably, handle 34 is approximately 2 cm to 3 cm long and has a greater diameter than shaft 39, as such a design may facilitate manipulation by the clinician.

When components 11, 20 and 30 are assembled, distal end 33 of plunger 30 is inserted into lumen 26 of inner tube 20, and distal end 23 of inner tube 20 is inserted into lumen 16 of outer tube 11. As depicted in FIG. 1, distal end 23 of inner tube 20 extends through opening 17 of outer tube 11. Markings 24 on body 29 of inner tube 20 permit the clinician to determine how far inner tube 20 extends beyond the distal end of outer tube 11 and also identifies the circumferential position of aperture 32. This configuration is an appropriate configuration to collect fluid, cells, and intrauterine samples.

Next, a preferred method of using device 10 of the present invention is described. First, the clinician inserts distal end 13 of outer tube 11 into the patient's vaginal canal. The clinician then advances outer tube 11 and positions applicator 12 against the patient's external cervical os. Once so positioned, the clinician may attach a conventional syringe containing anesthetic to Luer-Lok fitting 14 on proximal end 15 of outer tube 11. The clinician then may deliver the desired dosage of anesthetic, which passes through lumen 16 of outer tube 11 and to the cervical os. Applicator 12 reduces leakage of anesthetic into the vaginal space by directing the anesthetic toward the cervical os and acting as a barrier to retain the anesthetic in the uterus. Applicator 12 preferably remains in position for the duration of the sample collection procedure. At this point, either the patient or the clinician may continue to hold outer tube 11 in place until the anesthetic takes effect.

The clinician confirms that plunger 30 is positioned inside inner tube 20. Once sufficient time has passed for the anesthetic to take effect, the clinician removes the syringe from Luer-Lok fitting 14 and inserts distal end 23 of inner tube 20 into opening 18 at proximal end 15 of outer tube 11. The clinician then may refer to markings 24 and turn inner tube 20 so that aperture 22 is facing in the desired direction. Inner tube 20 and plunger 30 are advanced through lumen 16 until distal end 23 of inner tube 20 penetrates the patient's uterus and inner tube 20 extends beyond the distal end of outer tube 11. The clinician may the position aperture 22 so that it contacts the intrauterine lining to permit collection of intrauterine cells.

FIG. 3 schematically represents the general orientation of device 10 when positioned within a patient. Once inner tube 20 is oriented to the clinician's satisfaction, the clinician preferably holds inner tube 20 in place and retracts plunger 30 proximally. This movement of plunger 30 reduces the pressure in lumen 26 distal to end portion, causing intrauterine tissue and fluid to be drawn through aperture 22 and into lumen 26. The clinician then withdraws both inner tube 20 and plunger 30 in unison. Preferably, the clinician maintains the relative positions of inner tube 20 and plunger 30 during this step to prevent the tissue sample from becoming dislodged from lumen 26.

Once distal end 23 of inner tube 20 has been retracted back into outer tube 11, the clinician removes inner tube 20 from outer tube 11. At this point, the clinician may examine the contents of lumen 26 of inner tube 20 to determine whether sufficient sample has been removed from the patient. If not, inner tube 20 and plunger 30 may be reinserted and the procedure repeated until a sufficient sample is obtained. Once a satisfactory sample has been collected, the clinician removes outer tube 11 from the patient, thereby completing the tissue collection process.

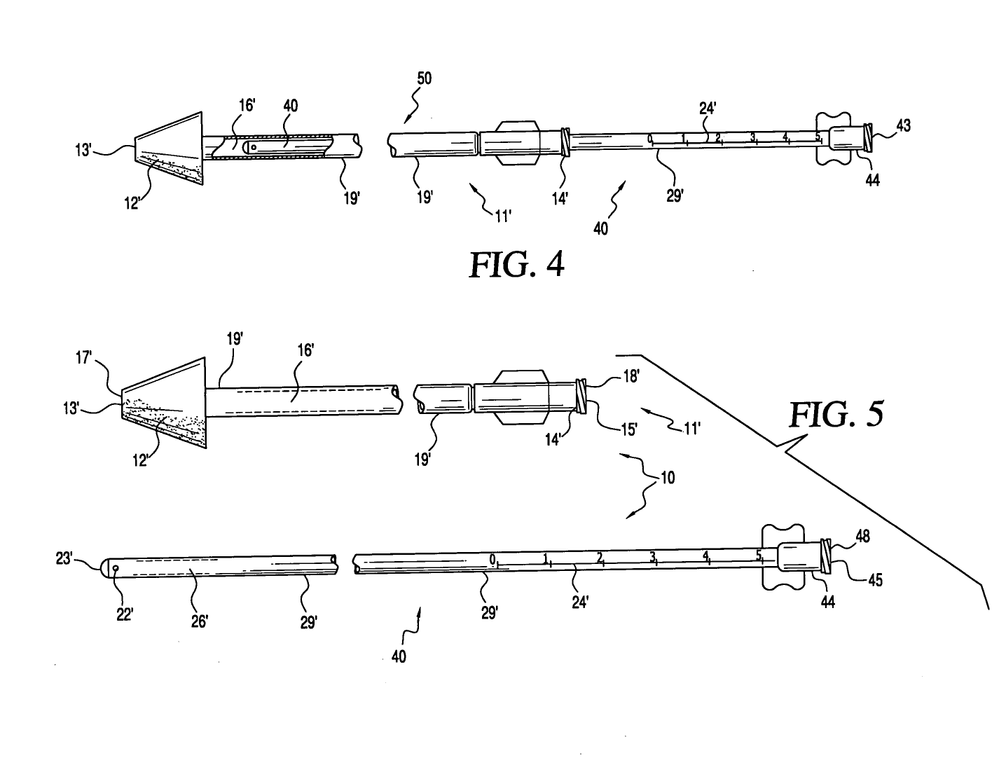

Referring now to FIGS. 4 and 5, an alternative embodiment of the present invention is described. Device 50 comprises outer tube 11′ and inner tube 40. Components of device 50 similar to the embodiment of FIGS. 1-3 are identified by like-primed numbers. Outer tube 11′ is similar in construction to outer tube 11 of the embodiment of FIGS. 1 and 2. Optionally, Luer-Lok fitting 14 of outer tube 11 may be omitted. Inner tube 40 also is similar in construction to inner tube 20 of the embodiment of FIGS. 1 and 2, but in the embodiment of FIGS. 4 and 5 includes Luer-Lok fitting 44 at proximal end 45. Fitting 44 permits inner tube 40 to be coupled to a conventional syringe (for use as a suction source), in which case no plunger is required.

Inner tube 40 preferably is cylindrical, at least 5 cm to 10 cm longer than outer tube 11′, and configured to freely reciprocate inside lumen 16′ of outer tube 11′. In a preferred embodiment, inner tube 40 has markings 24′ on body 29′ near proximal end 45 that enable the clinician to determine the circumferential position of aperture 22′. Markings 24′ also may be used to determine the depth of overall insertion of inner tube 40 or insertion of inner tube 40 relative to outer tube 11′.

A method of using device 50 is now described. First, the clinician inserts distal end 13′ of outer tube 11′ into a patient's vaginal canal. The clinician then advances outer tube 11′ and positions applicator 12′ against the patient's external cervical os. Anesthetic may then be applied by one of two methods. In a first method, the clinician delivers the anesthetic from a syringe attached to Luer-Lok fitting 14′ on proximal end 15′ of outer tube 11′.

In an alternative method, the clinician attaches a syringe containing anesthetic to the Luer-Lok fitting 44 on proximal end 45 of inner tube 40. Distal end 23′ of inner tube 40 then may be inserted into lumen 16′ of outer tube 11′ through opening 18′. Inner tube 40 is advanced through lumen 16′ until it is in the vicinity of the cervical os. The clinician then depresses the syringe to release the anesthetic through inner tube 40 and aperture 22′. Preferably, the clinician rotates inner tube 40 as the anesthetic passes through aperture 22′ to allow for a more uniform application.

Once the anesthetic is applied, either the patient or the clinician may continue to hold outer tube 11′ in place until the anesthetic takes effect. If the anesthetic was distributed directly through outer tube 11′, the clinician removes the syringe from Luer-Lok fitting 18′ and inserts inner tube 40 coupled to an empty syringe via Luer-Lok fitting 44. Inner tube 40 is inserted into opening 18′ and advanced until inner tube 40 is within the patient's uterus. If, instead, the anesthetic was applied via inner tube 40, the clinician advances inner tube 40 through lumen 16′ so that distal end 23′ is within the patient's uterus.

Once distal end 43 of inner tube 40 is within the patient's uterus, the clinician orients aperture 22′ by rotating inner tube 40 and observing markings 24′ on body 29′. Inner tube 40 is held in position by grasping Luer-Lok fitting 44 and then suction is applied to lumen 26′ of inner tube 40 by retracting the plunger of the syringe. This suction draws fluid and/or tissue from the uterine lining through aperture 22′ into lumen 26′ of inner tube 40. Preferably, suction is maintained while withdrawing inner tube 40 from lumen 16′ of outer tube 11′, so as to assist in separating the intrauterine tissue from the uterine wall. The clinician then completely withdraws inner tube 40 from outer tube 11′ to ensure sufficient sample was collected. If an insufficient sample was collected, the clinician may reinsert inner tube 40 and repeat the procedure. Once sufficient sample is collected, outer tube 11′ is removed from the patient.

The embodiment depicted in FIGS. 4 and 5 also may be used by a clinician to direct the application of the anesthetic to the precise site that the tissue sample will be taken. This directed application of anesthetic is accomplished after the initial application of anesthetic to the cervical os. Then, inner tube 40 is further advanced into the uterine cavity. Next, the clinician applies the anesthetic while noting markings 24′ to determine the position of inner tube 40. Finally, using markers 24′ to reposition inner tube 40 to the same location for the tissue collection, the clinician may position aperture 22′ at the site of localized anesthesia and proceed with the sample collection.

Although preferred illustrative embodiments of the present invention are described hereinabove, it will be evident to one skilled in the art that various changes and modifications may be made therein without departing from the invention. It is intended in the appended claims to cover all such changes and modifications that fall within the true spirit and scope of the invention.

What is claimed is:

1. Apparatus for applying medication to and collecting cell samples from a patient's uterus, the apparatus comprising:
an outer member having a distal end, a proximal end and a lumen extending therebetween, the outer member including a foam applicator disposed on the distal end;
an inner member having a distal end including a tissue collection opening, a proximal end and a lumen extending between the tissue collection opening and the proximal end, the inner member configured to reciprocate through the lumen of the outer member; and
means for drawing fluid and tissue through the opening into the lumen of the inner member.

2. The apparatus of claim 1 wherein the foam applicator has a conical shape.

3. The apparatus of claim 1 wherein the foam applicator conforms to the patient's cervical os.

4. The apparatus of claim 1, wherein the outer member further comprises a Luer-Lok fitting at the proximal end.

5. The apparatus of claim 1, wherein the inner member further comprises one or more markings near the proximal end.

6. The apparatus of claim 1, wherein the means for drawing fluid and tissue through the opening into the lumen of the inner member comprises a plunger configured to reciprocate through the lumen of the inner member.

7. The apparatus of claim 1, wherein the means for drawing fluid and tissue through the opening into the lumen of the inner member comprises a syringe coupled to the proximal end of the inner member via a Luer-Lok fitting.

8. The apparatus of claim 1, wherein outer member has a length in a range of 20 to 30 cm.

9. The apparatus of claim 8, wherein the inner member a length in a range of 5 to 10 cm longer than the length of the outer member.

10. The apparatus of claim 1 wherein the outer member and the inner member comprise polymeric materials.

11. Apparatus for applying medication to and collecting cell samples from a patient's uterus, the apparatus comprising:
an outer member having a distal end, a proximal end and a lumen extending therebetween, the outer member including a conical applicator disposed on the distal end;
an inner member having a distal end including a tissue collection opening, a proximal end and a lumen extending between the tissue collection opening and the proximal end, the inner member configured to reciprocate through the lumen of the outer member; and
means for drawing fluid and tissue through the opening into the lumen of the inner member.

12. The apparatus of claim 11 wherein the conical applicator comprises soft closed-cell foam.

13. The apparatus of claim 12 wherein the conical applicator conforms to the patient's cervical os.

14. The apparatus of claim 11, wherein the outer member further comprises a Luer-Lok fitting at the proximal end, the Luer-Lok fitting facilitating the delivery of anesthetic into the patient's uterus.

15. The apparatus of claim 11, wherein the inner member further comprises one or more markings near the proximal end.

16. The apparatus of claim 11, wherein the means for drawing fluid and tissue through the opening into the lumen of the inner member comprises a plunger configured to reciprocate through the lumen of the inner member.

17. The apparatus of claim 11, wherein the means for drawing fluid and tissue through the opening into the lumen of the inner member comprises a syringe coupled to the proximal end of the inner member via a Luer-Lok fitting.

18. The apparatus of claim 11, wherein outer member has a length in a range of 20 to 30 cm.

19. The apparatus of claim 18, wherein the inner member a length in a range of 5 to 10 cm longer than the length of the outer member.

20. The apparatus of claim 11 wherein the outer member and the inner member comprise polymeric materials.

21. A method for applying medication and collecting cell samples comprising:
providing apparatus having an outer member, an inner member, and a suction source, the outer member having a distal end, a proximal end and a lumen extending therebetween, the inner member having a distal end, a proximal end and a lumen extending therebetween, the inner member slidably disposed within the outer member and further having an opening disposed at or near the distal end;
inserting the distal end of the outer member into the patient;
delivering medication through the lumen of the outer member or the lumen of the inner member;
inserting the distal end of the inner member into the proximal end of the outer member;
extending the distal end of the inner member beyond the distal end of the outer member;
applying suction to the proximal end of the inner member to draw cells from the patient through the opening in the inner member; and
removing the apparatus from the patient.

22. The method of claim 21, wherein applying suction to the proximal end of the inner member comprises inserting a plunger within the lumen of the inner member and retracting the plunger to create suction.

23. The method of claim 21, wherein applying suction to the proximal end of the inner member comprises coupling a syringe to the proximal end of the inner member and retracting a plunger of the syringe.

24. The method of claim 21, wherein the medication is delivered through the lumen of the outer member.

25. The method of claim 21, wherein the medication is delivered through the lumen of the inner member.
